(19) United States
(12) Patent Application Publication
(10) Pub. No.: US 2026/0191655 A1
(43) Pub. Date: Jul. 9, 2026

(54) INTERVERTEBRAL DISC IMPLANTS HAVING OFFSET CENTER OF ROTATION

(71) Applicant: CENTINEL SPINE, LLC, West Chester, PA (US)

(72) Inventors: Edward J. McShane, III, Collegeville, PA (US); Timothy J. Bertone, Philadelphia, PA (US); Damian Heinz, West Brandywine Township, PA (US); Thierry Marnay, Montpellier (FR)

(21) Appl. No.: 19/410,445

(22) Filed: Dec. 5, 2025

Related U.S. Application Data
(60) Provisional application No. 63/728,974, filed on Dec. 6, 2024.
(60) Provisional application No. 63/728,982, filed on Dec. 6, 2024.
(60) Provisional application No. 63/728,964, filed on Dec. 6, 2024.

(51) Int. Cl. A61F 2/44; A61F 2/30

(52) U.S. Cl. CPC A61F 2/4425; A61F 2002/30649; A61F 2002/443

(57) ABSTRACT

Intervertebral disc implants including artificial disc replacement implants are provided for insertion into a disc space between two adjacent vertebrae. The implant comprises a first component having an outer surface for engaging one of the adjacent vertebrae and a concave surface opposite the outer surface and a second component having an outer surface for engaging the other of the adjacent vertebrae and an inner surface opposite the outer surface. The second component has a posterior end and an anterior end and a midline axis extending through the second component equally distant between the anterior and posterior ends. The implant further comprises an articulating core member residing between the first and second components and comprising a semi-spherical protrusion with a convex surface in contact with the concave surface of the first component. The apex of the convex surface is posterior to the midline axis of the second component.

### CROSS-REFERENCE TO RELATED APPLICATIONS

This application claims the benefit of U.S. Provisional Application Nos. 63/728,982, 63/728,974 and 63/728,964, all of which were filed on Dec. 6, 2024, the complete disclosures of which are incorporated herein by reference for all purposes.

### TECHNICAL FIELD

Implantable spinal implants, more specifically intervertebral disc implants, and even more specifically artificial disc replacement implants configured to accommodate the various orientations of the body structures in and around the intervertebral space.

### BACKGROUND

Spinal instability is often attributed to undesirable excessive motion between vertebrae which can cause significant pain and morbidity. The instability may result from a number of causes, including abnormalities of the vertebrae, the intervertebral discs, the facet joints, or connective tissue around the spine. These abnormalities may arise from diseases, disorders or defects of the spine from trauma or bone degradation, such as osteoarthritis, or degenerative disc disease. When the spine becomes unstable, the vertebral column becomes misaligned and may allow micromotion between adjacent vertebrae. Vertebral misalignment and micromotion may result in wear to the vertebral bone surfaces and ultimately generate severe pain. These conditions are often chronic and create progressive problems for the sufferer.

Known treatments for spinal instability can include long-term medical management or surgery. Medical management is generally directed at controlling the symptoms, such as pain reduction, rather than correcting the underlying problem. For some patients, this may require chronic use of pain medications, which may alter the patient's mental state or cause other negative side effects. Surgical treatment typically includes decompression procedures to restore normal disc height, realign the column, and alleviate the pain.

Today, a variety of implantable spinal stabilization devices are available to address these spinal conditions or abnormalities in a more permanent manner than with decompression alone. Some of these implantable devices include intervertebral spinal stabilization devices that are configured for placement between adjacent vertebrae. These devices generally fall under the category of either fusion-promoting, or motion-preserving, and may be configured for specific segments of the spine such as the thoracic, lumbar or cervical region of the spine.

One such type of motion-preserving intervertebral spinal stabilization device is the artificial disc implant. Artificial disc implants may be configured as a total artificial disc implant intended to replace the entire disc between two adjacent vertebrae, or a partial artificial disc implant that replaces only a portion while leaving a native remnant of the disc intact. The procedure, known as disc arthroplasty, involves the insertion of an artificial intervertebral disc implant into the intervertebral space between adjacent vertebrae. Such a disc implant allows limited universal movement of the adjacent vertebrae with respect to each other. The aim of total disc replacement is to remove pain generation (caused by a degenerated disc), restore anatomy (disc height), and maintain mobility in the functional spinal unit so that the spine remains in an adapted sagittal balance. Sagittal balance is defined as the equilibrium of the trunk with the legs and pelvis to maintain harmonious sagittal curves and thus the damping effect of the spine. In contrast with fusion techniques, total disc replacement preserves mobility in the motion segment and mimics physiologic conditions.

The biomechanical organization and overall shape of the intervertebral disc space may vary among individual patients. These varying parameters of individual anatomies may change the rotation mechanics of the disc implant. For example, the facet joints are symmetrical synovial-lined joints with a fibrous capsule that connect the articular facets of the vertebrae. The facet joints and the articular facets may have differing orientations or inclinations that create varying kinematics of anatomy between adjacent vertebrae. Biomechanics research indicates that the instantaneous Center of Rotation (COR) along the midline in the coronal plane during flexion and extension of the human spine. During this motion, in the sagittal plane, the COR lies near the posterior endplate of the disc space between the center of the vertebral endplate and posterior wall of the vertebral body normally. However, variations in human anatomy can change the position of the CORs. For example, if one or more of the facets are more inclined towards a vertical orientation than normal, the center of rotation of the functional spinal unit is translated more posteriorly toward the posterior border of the vertebral body. In addition, the radius of the rotation amidst flexion or extension can be smaller than normal. The current/normal obliquity of the facets and their “overlapping tile-like” relative positioning induces the combine motion of translation with rotation in flexion. A more vertical orientation of the facets leads to less translation at maximum flexion. This more vertical orientation of the facets induces a more posterior position of the center of rotation in the sagittal plane for flexion extension, and by consequence a smaller radius.

Thus, it would be desirable to provide disc replacement implants that accommodate the differing orientations of the vertebral structures and varied kinematics of adjacent vertebrae.

### SUMMARY

Intervertebral disc implants are provided that are configured for insertion within the disc space between two adjacent vertebral bodies. The implants may be configured for use anywhere along the spine as desired, including the cervical, lumbar or thoracic areas of the spine. These disc implants may be of the type that have an upper endplate, a lower endplate and an articulating core member therebetween that cooperate with one another to provide an articulating “ball in socket” joint between the endplates.

In one aspect, an implant for insertion into an intervertebral disc space between two adjacent vertebrae comprises a first component having an outer surface for engaging one of the adjacent vertebrae and a concave surface opposite the outer surface and a second component having an outer surface for engaging the other of the adjacent vertebrae and an inner surface opposite the outer surface. The implant further comprises an articulating core member residing between the first and second components and comprising a semi-spherical protrusion or dome with a convex surface in contact with the concave surface of the first component. The dome is eccentric relative to the first and/or the second component.

In various embodiments, the dome is positioned posteriorly to the center of the first and/or second components. In one such embodiment, the second component has a posterior end and an anterior end and a midline axis extending through the second component equally distant between the anterior and posterior ends. The dome comprises an apex that is posterior to the midline axis of the second component. Thus, dome is shifted in the posterior direction such that the COR is positioned closer to posterior end than the anterior end of the endplates. In certain embodiments, the implant is designed to accommodate a vertebral joint (or functional spinal unit) with a center of rotation (COR) naturally located more posteriorly, for example, when one or more of the facets are more inclined towards a vertical orientation than normal

In various embodiments, the convex surface of the dome defines a posterior surface area located posterior to the midline axis of the lower endplate and an anterior surface area located anterior to the midline axis of the lower endplate. The posterior surface area is greater than the anterior surface area. In an exemplary embodiment, the posterior surface area is about 52% to about 80%, or about 55% to about 75%, or about 55% to about 70%, of the total surface area of the convex surface of the dome.

In various embodiments, the convex surface of the semi-spherical dome defines an arc extending from a posterior end to an anterior end of the dome. The posterior end of the arc is closer to the posterior end of the lower endplate than the anterior end of the arc is to the anterior end of the lower endplate. In an exemplary embodiment, the posterior end of the arc is about 1 mm to about 5 mm, or about 2 mm to about 4 mm, from the posterior end of the lower endplate. The anterior end of the arc may be about 2 mm to about 8 mm, or about 4 mm to about 6 mm, from the anterior end of the lower endplate.

In an exemplary embodiment, the dome axis is spaced posteriorly from the midline axis by a distance of about 1 mm to about 10 mm, or about 2 mm to about 4 mm for cervical implants, and 2 about mm to 8 about mm for lumbar implants. Thus, the center of rotation (COR) is approximately 52-80% of the plate A-P depth of the inferior plate matched to the overall depth of the vertebral endplate.

In various embodiments, the outer surface of the upper endplate extends from an anterior end to a posterior end opposite the anterior end and the outer surface is continuously convex from the anterior end to the posterior end. In some embodiments, the outer surface of the upper endplate extends from a first lateral end to a second lateral end opposite the first lateral end and the outer surface is continuously convex from the first lateral end to the second lateral end. In an exemplary embodiment, the outer surface has a first radius of curvature from the anterior end to the posterior end and a second radius of curvature from the first lateral end to the second lateral end. The first radius of curvature is less than the second radius of curvature.

In various embodiments, the articulating core member comprises a semi-spherical member and an inlay. The inlay may be coupled to the lower implant and comprises a recess for receiving the semi-spherical member. Alternatively, the inlay and the semi-spherical member may be formed from a single integral component.

In various embodiments, the upper and lower endplates each comprise a fixation element extending from their outer surfaces. In one such embodiment, the fixation element(s) comprise one or more spikes. In other embodiments, the fixation elements comprise one or more keels. In yet another embodiment, the fixation elements comprise a combination of spikes and keels.

In various embodiments, the intervertebral disc space is a cervical intervertebral disc space.

In another aspect, an implant for insertion into an intervertebral disc space between two adjacent vertebrae comprises a first component having an outer surface for engaging one of the adjacent vertebrae and a concave surface opposite the outer surface and a second component having an outer surface for engaging the other of the adjacent vertebrae and an inner surface opposite the outer surface. The implant further comprises an articulating core member residing between the first and second components and comprising a semi-spherical protrusion with a convex surface having a first portion with a first surface area in contact with the concave surface of the first component and a second portion with a second surface area disposed between the first and second components (i.e., that does not directly contact the concave surface of the first component).

In various embodiments, the semi-spherical protrusion comprises a dome and the first component is an upper endplate. The upper endplate may comprise an internal cup having the concave surface for receiving the dome. The internal cup (or the internal concave surface of the upper endplate) is recessed or “sunken” far enough into the upper endplate that a greater percentage of the convex surface of the dome is in contact with the internal cup than is disposed between the endplates. This configuration positions the upper endplate closer to the lower endplate, thereby reducing the overall height of the implant.

In an exemplary embodiment, the first surface area of the convex surface of the dome that is in contact with the concave surface of the upper endplate comprises about 52% to about 80%, or about 55% to about 75% of the overall surface area of the convex surface.

In some embodiments, the ratio of the first surface area of the convex surface to the second surface area of the convex surface is greater than about 1.4 to 1, or greater than about 1.6 to 1, or greater than about 2 to 1.

In various embodiments, the upper endplate is configured to articulate relative to the lower endplate from a first “anatomically neutral position” to a second “rotated” position. The anatomically neutral position is generally defined as the position of the endplates when the two adjacent vertebrae are not rotated (in flexion/extension, lateral bending, or rotation) relative to each other (i.e., they are disposed in an anatomically neutral configuration). In some embodiments, the upper surface of the upper endplate may be parallel to the lower endplate in the anatomically neutral position. In other embodiments, the upper surface of the upper endplate may be oriented at an angle of 3-12 degrees, but can range from about 0 -20 degrees, in the anatomically neutral position. In these embodiments, a maximum distance between the outer surfaces of the first and second endplates in the anatomically neutral position is about 12 mm or less, or about 10.81 mm or less. A minimum distance between the outer surfaces of the first and second endplates in the anatomically neutral position is about 9.5 mm or less, or about 8.25 mm or less.

In various embodiments, the first component is an upper endplate and the second component is a lower endplate and the upper endplate comprises an inner surface facing the lower endplate. The inner surface of the upper endplate comprises first and second portions. The first portion extends at an angle relative to the second portion. The angle may be between 3-12 degrees, but can range from 0 -20 degrees.

In various embodiment, the first portion may comprise a posterior portion of the inner surface. This posterior portion tapers away from the lower endplate in the posterior direction. This configuration provides more room between the posterior portions of the upper and lower endplates, which increases the range of motion of the upper endplate, particularly in posterior rotation around an axis substantially perpendicular to the longitudinal axis of the implant.

In various embodiments, the posterior portion of the lower surface of the upper endplate contacts the inner surface of the upper endplate in a second position, which may represent the maximum posterior rotation of the upper endplate. The distance between the outer surfaces of the first and second endplates in the second position is less than about 8 mm, or about 6 mm.

In various embodiments, the articulating core member comprises a semi-spherical member and an inlay. The inlay may be coupled to the lower implant and comprises a recess for receiving the semi-spherical member. Alternatively, the inlay and the semi-spherical member may be formed from a single integral component.

In various embodiments, the upper and lower endplates each comprise a fixation element extending from their outer surfaces. In one such embodiment, the fixation element(s) comprise one or more spikes. In other embodiments, the fixation elements comprise one or more keels. In yet another embodiment, the fixation elements comprise a combination of spikes and keels.

In various embodiments, the intervertebral disc space is a cervical intervertebral disc space.

In various embodiments, the outer surface of the upper endplate extends from a first lateral end to a second lateral end opposite the first lateral end. The outer surface has a substantially continuous convex curve from the first lateral end to the second lateral end.

In various embodiments, the outer surface has a first radius of curvature from the anterior end to the posterior end and a second radius of curvature from the first lateral end to the second lateral end. The first radius of curvature is less than the second radius of curvature. The first radius of curvature may be substantially equal from the anterior end to the posterior end. The second radius of curvature may be substantially equal from the first lateral end to the second lateral end. The difference in curvature can allow for some change in position translationally of the articulating recessed plate relative to the inferior plate with dome as the upper plate rotates about the device's center.

In various embodiments, the ratio of the first radius of curvature to the second radius of curvature is about 1:2 to about 4:5, or about 2:3 to about 4:5. In certain embodiments the first radius of curvature may be between about spherical radius (SR) 5 mm to about SR 8 mm and the second radius of curvature may be between about SR 8 mm to about SR 10 mm for cervical applications. The first radius of curvature may be between about SR 14 mm to about SR 16 mm and the second radius of curvature may be between about SR 16 mm to about SR 20 mm for lumbar applications.

In various embodiments, the articulating core member comprises a semi-spherical member and an inlay. The inlay may be coupled to the lower implant and comprises a recess for receiving the semi-spherical member. Alternatively, the inlay and the semi-spherical member may be formed from a single integral component. The recess of the inlay may have a circular, or non-circular shape, such as an oblong, oval or egg shape.

In various embodiments, the upper and lower endplates each comprise a fixation element extending from their outer surfaces. In one such embodiment, the fixation element(s) comprise one or more spikes. In other embodiments, the fixation elements comprises one or more keels. In yet another embodiment, the fixation elements comprise a combination of spikes and keels.

In various embodiments, the intervertebral disc space is a cervical intervertebral disc space.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a perspective view of a spinal implant;

FIG. 2 is a side view of the spinal implant of FIG. 1;

FIG. 3 is a cross-sectional side view of the bottom portion of the spinal implant of FIG. 1;

FIG. 4A is a perspective view of a spinal implant;

FIG. 4B is a top view of the spinal implant of FIG. 4A;

FIG. 4C is a cross-sectional side view of the spinal implant of FIG. 5;

FIG. 5A is a back view of the spinal implant of FIG. 4A

FIG. 5B is a cross-sectional front view of the spinal implant of FIG. 4B

FIG. 6 is side view of a spinal implant;

FIG. 7 is a side view of the spinal implant of FIG. 6 rotated in extension with the upper portion rotated in the posterior direction;

FIG. 8 is a side view of the spinal implant of FIG. 6 rotated in flexion with the upper portion rotated in the anterior direction;

FIG. 9A is a perspective view of a spinal implant;

FIG. 9B is a side view of the spinal implant of FIG. 9A;

FIG. 10A is a perspective view of a spinal implant;

FIG. 10B is a side view of the spinal implant of FIG. 10A;

FIG. 11A is a side view of a cervical spine taken in the sagittal plane;

FIG. 11B is an enlarged sagittal plane view of superior and inferior facet processes for one of the intervertebral spaces of the cervical spine in a standard orientation; and

FIG. 11C is an enlarged sagittal plane view of superior and inferior facet processes in a more vertical orientation.

### DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a”, “an”, and “the”, and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Spinal devices are provided that are configured for implantation within an intervertebral space disposed between vertebral bodies. The vertebral bodies can be anatomically adjacent vertebral bodies, or can be the vertebral bodies that remain after a discectomy has been performed that removed a vertebral body from a location between the vertebral bodies. The devices are configured to be inserted into the intervertebral space, and achieve restoration of height while maintaining mobility. The intervertebral space can be disposed anywhere along the spine as desired, including the cervical, lumbar or thoracic areas of the spine. The implants described herein are particularly useful for conforming a specific implant to a particular anatomical space, thereby increasing the ability of the implant to duplicate the natural movement of the spine.

Certain terminology is used in the following description for convenience only and is not limiting. The words “right”, “left”, “lower” and “upper” designate directions in the drawings to which reference is made. The words “inner” or “distal” and “outer” or “proximal” refer to directions toward and away from, respectively, the geometric center of the implant and related parts thereof. The words “anterior”, “posterior”, “superior”, “inferior”, “medial”, “lateral”, and related words and/or phrases designate preferred positions and orientations in the human body to which reference is made and are not meant to be limiting. The terminology includes the above listed words, derivatives thereof and words of similar import.

Unless otherwise specified herein, the terms “lateral”, “longitudinal”, and “transverse” are used to describe the orthogonal directional components of various components. It should be appreciated that while the longitudinal and lateral directions are illustrated as extending along a horizontal plane, and that the transverse direction is illustrated as extending along a vertical plane, the planes that encompass the various directions may differ during use. For instance, when the implant is implanted into an intervertebral space, such as the intervertebral space, the transverse direction extends generally along the superior-inferior (or caudal-cranial) direction, while the plane defined by the longitudinal direction and lateral directions generally in the anatomical plane defined by the anterior-posterior direction, and the medial-lateral direction. Accordingly, the directional terms “vertical” and “horizontal” are used to describe the implants and their components as illustrated merely for the purposes of clarity and illustration.

For purposes of clarity, the longitudinal axis is hereinafter defined as the axis generally extending from an anterior portion of the implant to a posterior portion of the prosthesis after insertion of the implant into the intervertebral body. The anterior portion may also be considered the trailing end of the prosthesis and the posterior portion the leading end (with reference to the direction the prosthesis is introduced into the intervertebral space). The lateral axis is hereafter defined as the axis perpendicular to the longitudinal axis and generally extending from one lateral side of the implant to the other lateral side.

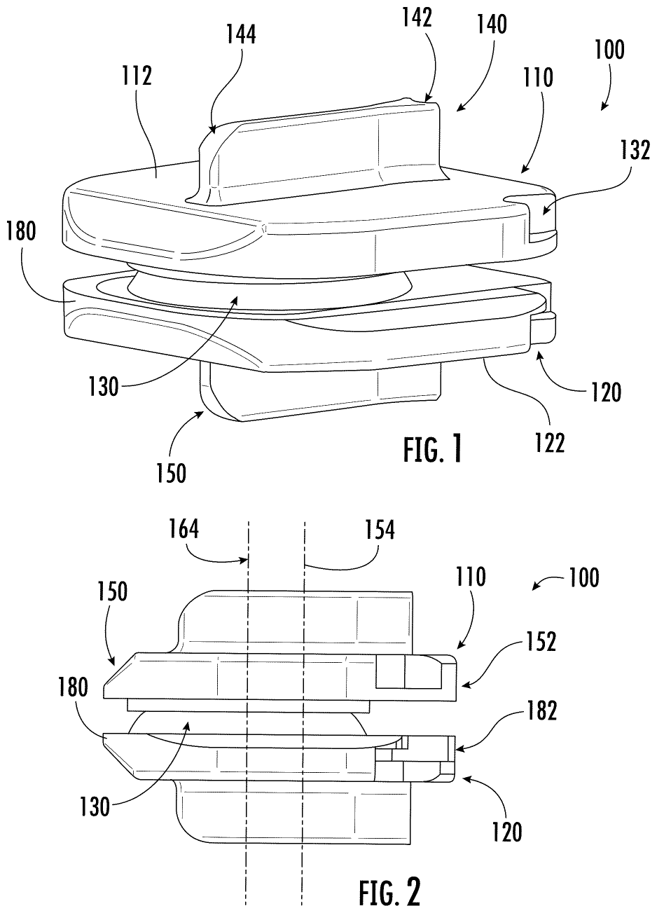

Referring now to FIGS. 1-3, a spinal implant 100 comprises an upper or superior endplate 110, a lower or inferior endplate 120 and a core 130. Superior endplate 110 rides upon the core 130 and is operable to rotate relative to core 130, including torsional rotation about a vertical axis, flexion/extension rotation in the posterior/anterior directions and lateral bending rotation. Core 130 includes a substantially spherical dome 160 having a convex upper surface 165 that contacts an internal concave surface (not shown) of upper endplate 110 and an inlay 170 that couples dome 160 to lower endplate 120. The spherical dome 160 may be a separate element from the inlay 170 and coupled thereto, or the core and inlay may be manufactured as a single, integral element.

Core 130 may be coupled to lower endplate 120 in any suitable manner. In an exemplary embodiment, dome 160 is coupled to an upper recess within inlay 170 and inlay 170 is slidably disposed within longitudinal recesses or slots (not shown) in lower endplate 110. For example, inlay 170 may include a plastic snap-in projection that fits within snap-in recesses or slots in lower endplate 120 such that the plastic inlay can snap into place but is thereafter inhibited from being removed. The recess of the inlay may have a round or circular shape. In some embodiments, the recess of the inlay may have a non-round or non-circular shape, such as an oblong, oval or egg shape.

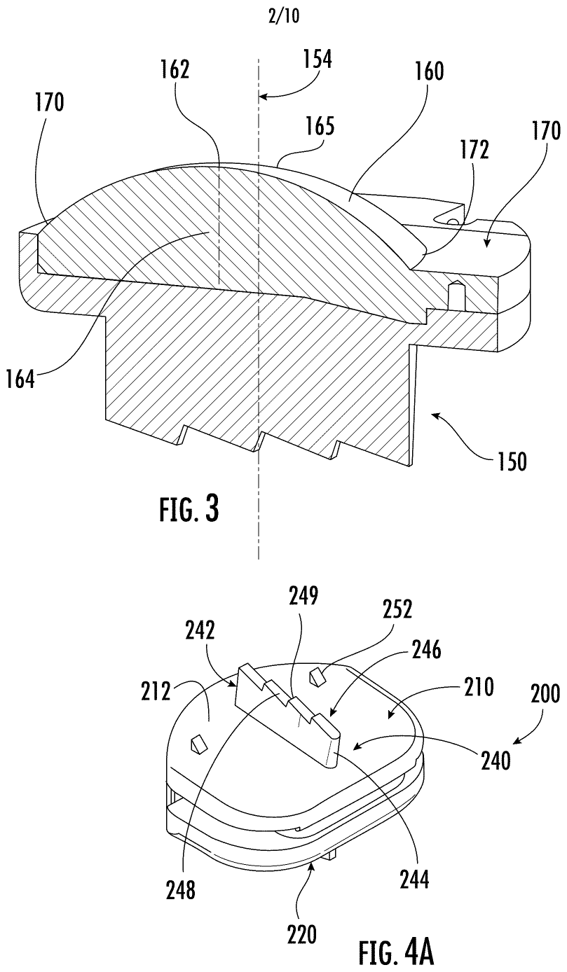

Core 130 is eccentric or not-centered relative to upper and lower endplates 110, 120. Endplates 110, 120 have a leading or posterior end 150, a trailing or anterior end 152 and an axis 154 extending through a midline of the endplates approximately halfway between posterior and anterior ends 150, 152. In this embodiment, core 130 is shifted in the posterior direction such that the core 130 is positioned closer to posterior end 150 than anterior end 152. In particular, spherical dome 160 has an apex 162 that defines an axis or radius 164 extending from apex 162 to a theoretical center of the spherical dome 160 (see FIGS. 2 and 3). The axis 164 is spaced in a posterior direction from midline axis 154. In an exemplary embodiment, dome axis 164 is spaced posteriorly from midline axis 154 by a distance of about 1 mm to about 10 mm, or about 2 mm to about 4 mm for cervical implants, and about 2 mm to 8 about mm for lumbar implants. Thus, the center of rotation (COR) is approximately 52-80% of the plate A-P depth of the inferior plate matched to the overall depth of the vertebral endplate.

Convex upper surface 165 of dome 160 generally has a posterior surface area located on the posterior side of midline axis 154 and an anterior surface area located on the anterior side of midline axis 154. The posterior surface area is greater than the anterior surface area. In an exemplary embodiment, the posterior surface area of surface 165 is about 52% to about 80%, or about 55% to about 75%, or about 55% to about 70%, of the anterior surface area of surface 165.

Convex upper surface 165 of dome 160 defines an arc that generally extends from a posterior or leading end 170 of dome 160 to an anterior or trailing end 172 of dome 160. The posterior end of the arc is closer to the posterior end of the lower endplate than the anterior end of the arc is to the anterior end of the lower endplate. In an exemplary embodiment, posterior end 170 of surface 165 is about 1 mm to about 5 mm, or about 2 mm to about 4 mm, from a posterior end surface 180 of lower endplate 120. Anterior end 172 of surface 165 is about 2 mm to about 8 mm, or about 4 mm to about 6 mm from an anterior end surface 182 of lower endplate 120.

In one embodiment, the upper bone contacting surface 112 of upper endplate 110 is substantially flat and the side surfaces of upper endplate 110 are substantially perpendicular to the upper surface and include rounded or beveled corners. Similarly, the lower bone contacting 122 surface of lower endplate 120 is substantially flat and the side surfaces of lower endplate 120 are substantially perpendicular to the lower surface and include rounded or beveled corners. In some embodiments, upper and/or lower endplates 110, 120 have recesses or notches 132 carved into the two anterior corners.

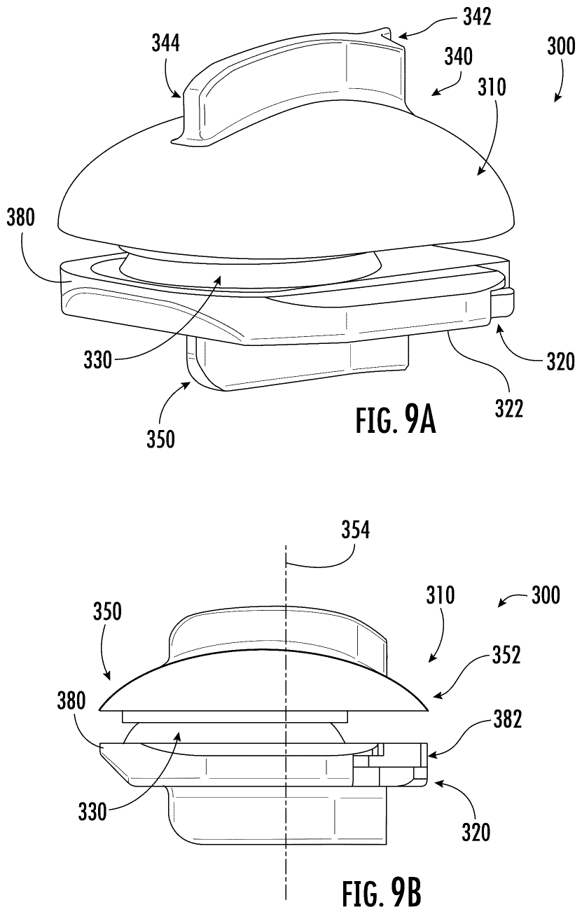

In other embodiments, bone contacting surface 112 (and/or surface 122) may have a substantially domed shape such that part of, or the entire, contact surface is curved. In some embodiments, the contact surfaces may be generally convex from an anterior end of each surface to the posterior end of each surface. Similarly, the contact surfaces may be generally convex from a first lateral end of the surface to a second lateral end opposite the first lateral end. In some embodiments, upper bone contacting surface 112 may have the same general surface as shown in FIGS. 9A and 9B.

Implant 100 further includes fixation elements extending from each of the bone contacting surfaces 112, 122. These fixation elements may comprise one or more keels and/or one or more spikes and may have any of the configurations described herein. Implant 100 further includes a first keel 140 extending from upper bone contacting surface 112 of upper endplate 110 and a second keel 150 extending from lower bone contacting surface 122 of lower endplate 120. Keels 120, 140 are centrally located on endplates 110, 120 such that they generally extend along a longitudinal axis of the endplates.

Keels 140, 150 each include an anterior or trailing end 142 that flares laterally outward to anchor this end of the keels in their cutouts in adjacent vertebrae. Keels 140, 150 each extend along the bone contacting surfaces such that a space exists between the anterior and posterior surfaces of the bone contacting surfaces of endplates 110, 120 and the keels. The sides surfaces of keels 140, 150 are substantially perpendicular to the bone contacting surfaces of endplates 140, 150, but, in some embodiments, may also taper inward in the directions away from these bone contacting surfaces such that the upper portion of keels 140, 150 has a smaller width than the base. The top surfaces of keels 140, 150 are preferably rounded or beveled to create a smooth curved transition from the side surfaces to the top surfaces. The posterior or leading end 144 of the keels 140, 150 are beveled to facilitate insertion of the keels into cutouts formed in the adjacent vertebrae. A more complete description of suitable keels can be found in commonly assigned, U.S. Pat. No. 8,998,990, the complete disclosure of which is incorporated herein by reference for all purposes.

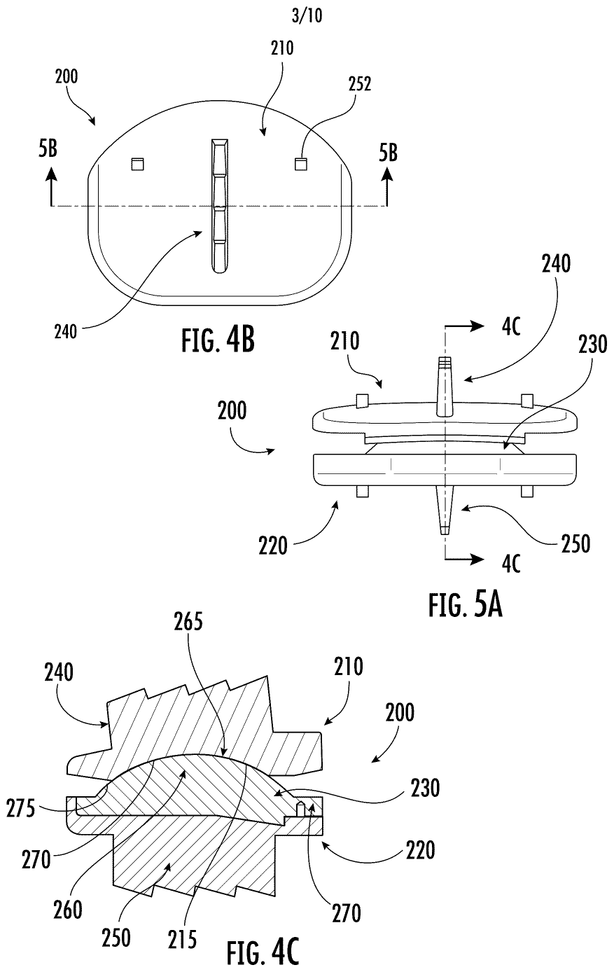

In an alternative embodiment, keels 140, 150 may have a serrated or jagged outer surface such as that shown in FIG. 3 and FIGS. 4A-4C, as described in more detail below.

Upper and lower endplates 110, 120 and components thereof, can be formed of any suitable biocompatible material, such as cobalt chromium molybdenum (CoCrMo), titanium and titanium alloys, stainless steel, ceramics, or polymers such as polyetheretherketone (PEEK), polyetherketoneketone (PEKK), bioresorbable materials, and bone graft (for example, allografts and xenografts). A coating may be added or applied to the endplates to improve physical or chemical properties. The coatings may help to ensure bony in or on growth or medication. Examples of coatings include plasma-sprayed titanium coating or hydroxyapatite. The porosity of the coating ideally permits vascularization and osteoblast formation with subsequent bony on-growth.

In use, implant 100 may be introduced into the intervertebral space between two adjacent vertebrae in the cervical or lumbar regions of the spine to replace all or a portion of the natural disc. The procedure is generally known as disc arthroplasty. The biomechanical organization and overall shape of the intervertebral disc space, however, may vary among individual patients. These varying parameters of individual anatomies may change the rotation mechanics of the disc implant 100. For example, the facet joints are symmetrical synovial-lined joints with a fibrous capsule that connect the articular facets of the vertebrae. The facet joints and the articular facets may have differing orientations or inclinations that create varying kinematics of anatomy between adjacent vertebrae. Biomechanics research indicates that the instantaneous Center of Rotation (COR) occurs along the midline in the coronal plane during flexion and extension of the human spine. During this motion, in the sagittal plane, the COR lies near the posterior endplate of the disc space between the center of the vertebral endplate and posterior wall of the vertebral body normally. However, variations in human anatomy can change the position of the CORs. For example, if one or more of the facets are more inclined towards a vertical orientation than normal, the center of rotation of the functional spinal unit is translated more posteriorly toward the posterior border of the vertebral body. In addition, the radius of the rotation amidst flexion or extension can be smaller than normal. The current/normal obliquity of the facets and their “overlapping tile-like” relative positioning induces the combined motion of translation with rotation in flexion. A more vertical orientation of the facets leads to less translation at maximum flexion. This more vertical orientation of the facets induces a more posterior position of the center of rotation in the sagittal plane for flexion extension, and by consequence a smaller radius.

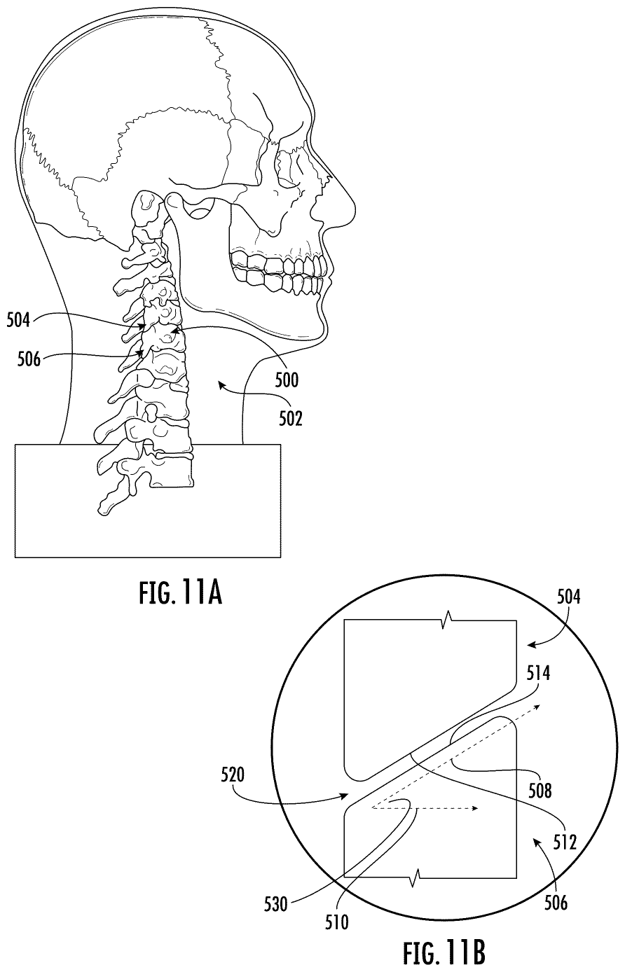

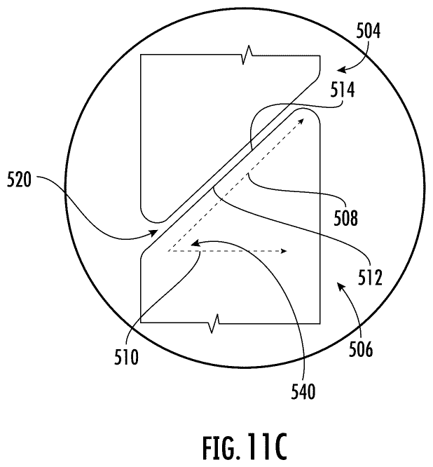

FIG. 11A illustrates a representative cervical region of the spine 502 with a plurality of intervertebral disc spaces 500, each including a superior facet process 504 and an inferior facet process 506. As shown in FIG. 11B, the normal or standard orientation of these processes is along an axis 508 that is oriented at an angle 530 of about 30 degrees to about 35 degrees relative to a horizontal axis 510 extending along the sagittal plane. The sagittal plane is a vertical plane that divides the body into left and right sections. Thus, a lower surface 512 of superior facet process 504 and an upper surface 514 of inferior facet process 506 may be substantially parallel to each other and oriented in a direction along axis 508. In some anatomies, there may be a gap 520 between surfaces 512, 514.

In certain anatomies, however, the orientation of these processes may be more vertical, such that the processes are oriented at an angle greater than 30-45 degrees relative to the horizontal axis 510 in the sagittal plane. FIG. 11C illustrates an example wherein the upper and lower surfaces 512, 514 of the facet processes 504, 506 are oriented at a “more vertical” angle 540 along an axis 508 relative to a horizontal axis 510. Angle 540 may be, for example, 35 degrees or greater, 40 degrees or greater or even 45 degrees or greater. In this configuration, the center of rotation of the functional spinal unit is translated more posteriorly toward the posterior border of the vertebral body. In addition, the radius of the rotation amidst flexion or extension can be smaller than normal. A more vertical orientation of the facets leads to less translation at maximum flexion. This more vertical orientation of the facets induces a more posterior position of the center of rotation in the sagittal plane for flexion extension, and by consequence a smaller radius.

In a method of use, a user may select an implant based on the orientation of the facets. For example, in the event that the orientation is determined to be “more vertical”, such that the angle of surfaces 512, 514 of facet processes 504, 506, is greater than the normal or standard orientation (e.g., 35-45 degrees or greater relative to the horizontal axis 510 in the sagittal plane as shown in FIG. 11C), the user may select an implant having a COR that is shifted in the posterior direction relative to a centered COR. As discussed above, implant 100 has a core 130 that is shifted in the posterior direction such that the core 130 is positioned closer to posterior end 150 than anterior end 152. Selecting such an implant will accommodate the more vertical orientation of the facet processes that shifts the COR of the natural implant posteriorly.

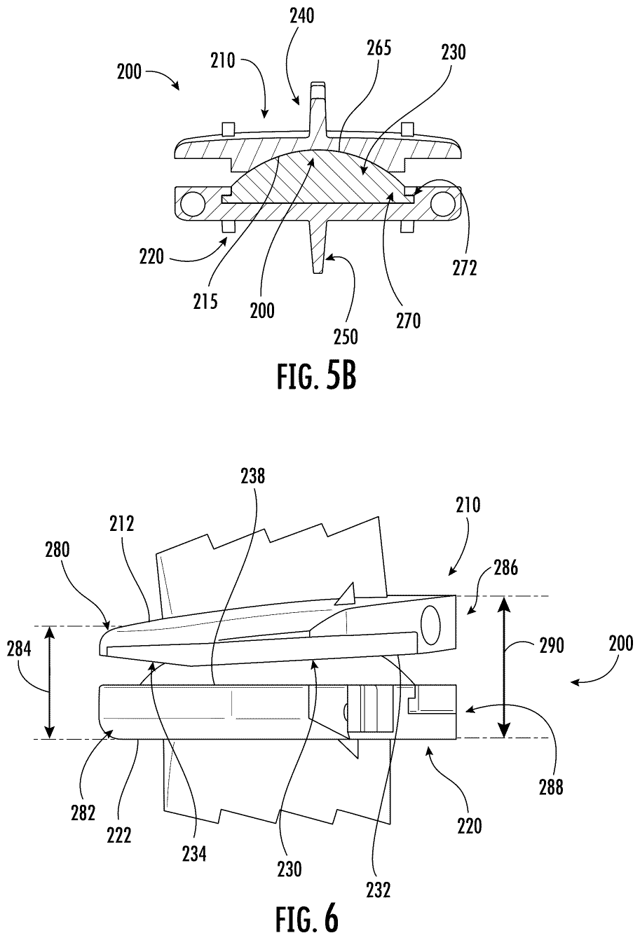

Referring now to FIGS. 4A-4C, 5A and 5B, a spinal implant 200 comprises an upper or superior endplate 210, a lower or inferior endplate 220 and a core 230. Superior endplate 210 rides upon the core 230 and is operable to rotate relative to core 230, including torsional rotation about a vertical axis, flexion/extension rotation in the posterior/anterior directions and lateral bending rotation. Core 230 includes a substantially spherical dome 260 having a convex upper surface 265 that contacts an internal concave surface 215 of upper endplate 210 and an inlay 270 that couples dome 260 to lower endplate 220. The spherical dome 260 may be a separate element from the inlay 270 and coupled thereto, or the core and inlay may be manufactured as a single, integral element.

Core 230 may be coupled to lower endplate 220 in any suitable manner. In an exemplary embodiment, dome 260 is coupled to an upper recess within inlay 270 and inlay 270 is slidably disposed within longitudinal recesses or slots 272 in lower endplate 220. For example, inlay 270 may include a plastic snap-in projection that fits within snap-in recesses or slots in lower endplate 220 such that the plastic inlay can snap into place but is thereafter inhibited from being removed. The recess of the inlay may have a round or circular shape. In some embodiments, the recess of the inlay may have a non-round or non-circular shape, such as an oblong, oval or egg shape.

Implant 200 further includes fixation elements extending from each of the bone contacting surfaces 212, 222. These fixation elements may comprise one or more keels and/or one or more spikes and may have any of the configurations described herein. In the representative embodiment, implant 200 further includes a first keel 240 extending from upper bone contacting surface 212 of upper endplate 210 and a second keel 250 extending from lower bone contacting surface 222 of lower endplate 220. Keels 220, 240 are centrally located on endplates 210, 220 such that they generally extend along a longitudinal axis of the endplates.

Keels 240, 250 generally define a posterior or leading end 242 and an anterior or trailing end 244. Posterior and anterior ends 242, 244 may be substantially flat and perpendicular to bone contacting surfaces 212, 222, or they may be rounded or beveled. In certain embodiments, posterior or leading end 242 is rounded or beveled. Keels 240, 250 may have substantially flat or perpendicular side surfaces, or these surfaces may be tapered, or they may be rounded or beveled. In certain embodiments, keels 240, 250 have a segmented surface 246 opposite the bone contacting surfaces 212, 222 of endplates 210, 220. This surface 246 may comprise one or more segments 248 that each taper inwardly towards the endplates in the anterior or trailing direction such that the surface 246 of each segment 248 contacts a transverse surface 249 of the next segment 248. Keels 240, 250 may each include two, three or four or more segments 248. In some embodiments, keel 240 will have a different number and configuration of segments 248 than keel 250.

In certain embodiments, implant 200 may further include one or more spikes 252 projecting from bone contacting surfaces 212, 222. In one such embodiment, implant 200 includes one or more spikes in a first lateral region of the endplate body and one or more spikes in a second lateral region of the endplate body opposite the longitudinal axis from the first group of spikes. The spikes may be arranged in the posterior and/or anterior regions of the endplates. In one such embodiment, each endplate includes spikes in the posterior region of the endplates, as shown in FIG. 4A.

In certain embodiments, each spike has a substantially pyramidal shape and extends up from a base having a triangular or alternatively shaped footprint at the bone facing surface, to an upper or outer transverse tip. Each surface extends between the base and the tip, and can be connected between the base and the tip as illustrated. The spikes thus defines a transverse axis that extends transversely between the outer tip and the bone facing surface. The spikes define recesses therein that extend from a portion of spikes between the base and the tip and into, but not through, endplates.

Referring now to FIGS. 5A and 5B, concave surface 215 of upper endplate 210 is recessed or “sunken” into endplate 210 such that a greater portion of dome 260 resides within endplate 210 than resides between the endplates. As discussed below, this configuration allows upper endplate 210 to be disposed closer to lower endplate 220, which reduces the overall height of implant 200. In particular, convex surface 265 of dome 260 comprises a first portion 270 with a first surface area in contact with concave surface 215 of upper endplate 210 and a second portion 275 with a second surface area disposed between first and second endplates 210, 220. The second portion 275 generally will not be in contact with concave surface 215 when the endplates 210, 220 are in an anatomically neutral position. An anatomically neutral position is generally defined as the position of the endplates when the two adjacent vertebrae are not rotated (in flexion/extension, lateral bending, or rotation) relative to each other (i.e., they are disposed in an anatomically neutral configuration). In this position, upper endplate 210 may be substantially parallel to lower endplate 220, or it may be angled slightly in the posterior direction, as shown in FIG. 6. In any such event, upper endplate 210 will have an angle between about 0 degrees to about 20 degrees or about 3 degrees to about 12 degrees relative to lower endplate 220 in the anatomically neutral position.

The surface area of first portion 270 is greater than the surface area of second portion 275 because concave surface 215 is further recessed into endplate 210, as discussed above. This configuration “moves” upper endplate 210 downward and closer to lower endplate 220, thereby reducing the overall height of implant 200. In certain embodiments, the surface area of first portion 270 comprises about 52% to about 80%, or about 55% to about 75% of the overall surface area of convex surface 265. In various embodiments, the ratio of the surface area of first portion 270 to the surface area of second portion 275 is greater than about 1.4 to 1, or greater than about 1.6 to 1, or greater than about 2 to 1.

It should be appreciated that upper endplate 210 is configured to rotate relative to dome 260, and the numerical relationships described above correspond to the anatomically neutral position. In some patient anatomies, upper endplate 210 may be substantially parallel to lower endplate 220 in this neutral position, while in others it may be oriented at a slight posterior or anterior angle. Accordingly, the “first portion” 270 of convex surface 265 may be understood as the region of convex surface 265 that is in contact with concave surface 215 at a given time, whereas the “second portion” 275 refers to the region of convex surface 265 that is not in contact with concave surface 215. As upper endplate 210 moves or rotates relative to dome 260 during flexion, extension, or lateral bending, the specific areas defining the first and second portions may shift. However, the overall ratio between the surface area of convex surface 265 that is in contact with concave surface 215 and the surface area that is not in contact will remain substantially constant.

For instance, in an anatomically neutral configuration, approximately 60% of convex surface 265 may be in contact with concave surface 215 (first portion 270), while about 40% remains spaced apart (second portion 275). During flexion or extension, the points of contact may migrate along the dome surface, yet the general proportion of contact and non-contact regions remains within the same relative range.

As discussed above, the overall height of implant 200 is reduced as a result of the sunken cup. As shown in FIG. 6, implant 200 has a height or distance 284 between the posterior end 280 of surface 212 and the posterior end 282 of surface 222 of less than about 9.5 mm, or about 8.25 mm. Implant 200 has a height or distance 290 between the anterior end 286 of surface 212 and the anterior end 288 of surface 222 of less than about 12.4 mm, or about 10.81 mm. These distances can be measured when implant 200 is positioned in the anatomically neutral position, as defined above.

In certain embodiments, concave surface 215 may be part of a separate internal cup (not shown) that is coupled to upper endplate 210. In these embodiments, upper endplate 210 may have a lower surface that extends around the internal cup with the cup extending downward from this lower surface so that at least part of the internal cup is disposed between the endplates.

As discussed above, the recessed or sunken concave surface 215 of upper endplate 210 generally reduces the distance between the inner surfaces of the endplates. As a result of this, the overall range of motion of upper endplate 210 may be reduced relative to lower endplate 220 (i.e., because there is less room between the posterior/anterior ends and the opposing lateral ends of the endplates which reduces the degree of rotation of upper endplate 210). To offset this potential drawback, upper endplate 210 comprises an inner surface 230 having a first anterior portion 232 and a second posterior portion 234 disposed at an angle relative to anterior portion 232 (see FIG. 6). In particular, posterior portion 234 tapers away from inner surface 238 of lower endplate 220 in the posterior direction. In an exemplary embodiment, posterior portion 234 extends at an angle of about 0 degrees to about 20 degrees, or about 3 degrees to about 12 degrees, relative to anterior portion 232.

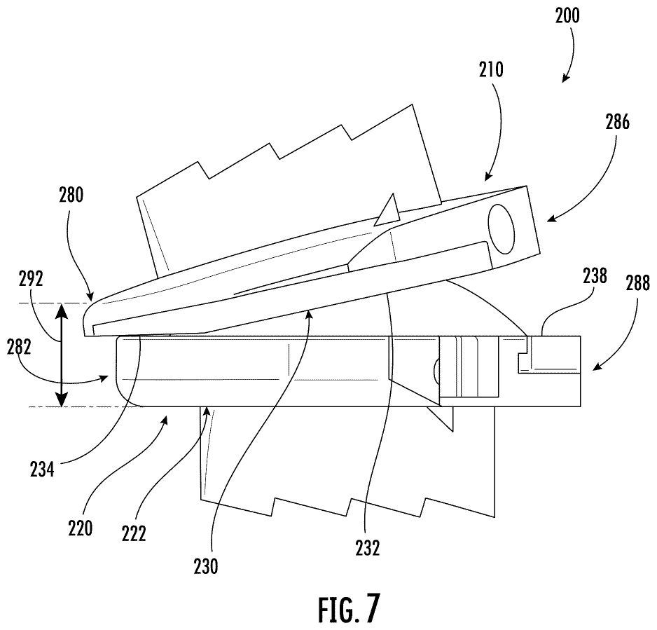

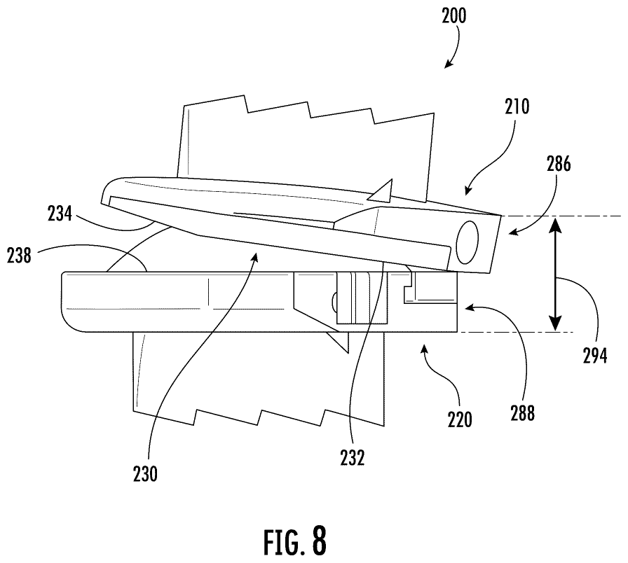

Referring now to FIG. 7, posterior portion 234 of inner surface 230 allows upper endplate 210 a greater degree of rotation in the posterior direction (i.e., around the axis extending into the page or perpendicular to a longitudinal axis of the implant) because posterior portion 234 tapers away from inner surface 238 of lower endplate 220. In particular, upper endplate 210 may rotate posteriorly into a position wherein posterior portion 234 contacts inner surface 238 as shown in FIG. 7. In this position, the distance or height 292 between the posterior end 280 of surface 212 and the posterior end 282 of surface 222 is less than about 8 mm, or less than about 7 mm, or about 6.02 mm.

Referring now to FIG. 8, the overall height or thickness of anterior portion 286 of upper endplate 210 has been reduced. Surface 230, when angled up, decreases the height of surface 286—which shortens the overall construct height and restores most of the available angulation in flexion. This provides a greater degree of rotation in the anterior direction despite the fact that upper endplate 210 is disposed closer to lower endplate 220. In particular, upper endplate 210 may rotate anteriorly into a position wherein anterior portion 232 contacts inner surface 238 of lower endplate 220 as shown in FIG. 8. In this position, the distance or height 294 between the anterior end 286 of surface 212 and the anterior end 288 of surface 222 is less than about 9 mm, or less than about 8 mm, or about 7.85 mm.

Referring now to FIGS. 9A and 9B, a spinal implant 300 comprises an upper or superior endplate 310, a lower or inferior endplate 320 and a core 330. Superior endplate 310 rides upon the core 330 and is operable to rotate relative to core 330, including torsional rotation about a vertical axis, flexion/extension rotation in the posterior/anterior directions and lateral bending rotation. Core 330 includes a substantially spherical dome 360 having a convex upper surface 365 that contacts an internal concave surface (not shown) of upper endplate 310 and an inlay 370 that couples dome 360 to lower endplate 320. The spherical dome 360 may be a separate element from the inlay 370 and coupled thereto, or the core and inlay may be manufactured as a single, integral element.

Endplates 310, 320 have a leading or posterior end 350, a trailing or anterior end 352 and an axis 354 extending through a midline of the endplates approximately halfway between posterior and anterior ends 350, 352 (see FIG. 9B). Similar to the embodiment shown in FIGS. 1-3, core 330 is shifted in the posterior direction such that the core 330 is positioned closer to posterior end 350 than anterior end 352. In particular, spherical dome 360 has an apex (not shown) that defines an axis or radius extending from the apex to a theoretical center of the spherical dome 360. This axis is spaced in a posterior direction from midline axis 354.

In this embodiment, upper endplate 310 has an upper bone contacting surface 312 that is substantially convex throughout the entire surface. Thus, surface 312 extends, and is substantially continuously convex, from its posterior end to its anterior end. In addition, surface 312 extends, and is substantially continuously convex, from a first lateral end to second lateral end. “Substantially continuous” refers to a surface configuration that maintains a generally smooth and uninterrupted curvature over its extent, such that no distinct discontinuities, sharp transitions, or planar regions are present that would materially alter the overall convex contour. Minor variations, surface textures, or manufacturing tolerances that do not affect the functional continuity of the curve are considered within the scope of “substantially continuous.”

Convex surface 312 has a first radius of curvature from the anterior end to the posterior end and a second radius of curvature from the first lateral end to the second lateral end. The second radius of curvature is greater than the first radius of curvature such that convex surface 312 extends downwardly towards lower endplate 320 more rapidly in the lateral direction than in the longitudinal direction. In addition, the convex surface 312 extends for a longer distance in the longitudinal direction than in the lateral direction. This creates a domed shape that substantially resembles a turtle shell.

In an exemplary embodiment, the ratio of the first radius of curvature to the second radius of curvature is about 1:2 to about 4:5, or about 2:3 to about 4:5. In certain embodiments the first radius of curvature may be between about spherical radius (SR) 5 mm to about SR 8 mm and the second radius of curvature may be between about SR 8 mm to about SR 10 mm for cervical applications. The first radius of curvature may be between about SR 14 mm to about SR 16 mm and the second radius of curvature may be between about SR 16 mm to about SR 20 mm for lumbar applications.

Similar to the embodiment shown in FIGS. 5A and 5B above, implant 300 may further include a recessed or sunken concave surface (not shown) of upper endplate 310 such that a greater portion of dome 360 resides within endplate 310 than resides between the endplates. As discussed below, this configuration allows upper endplate 310 to be disposed closer to lower endplate 320, which reduces the overall height of implant 300.

Implant 300 further includes fixation elements extending from each of the bone contacting surfaces 312, 322. These fixation elements may comprise one or more keels and/or one or more spikes and may have any of the configurations described herein. In the representative embodiment, implant 300 comprises a first keel 340 extending from upper bone contacting surface 312 of upper endplate 310 and a second keel 350 extending from lower bone contacting surface 322 of lower endplate 320. Keels 320, 340 are centrally located on endplates 310, 320 such that they generally extend along a longitudinal axis of the endplates.

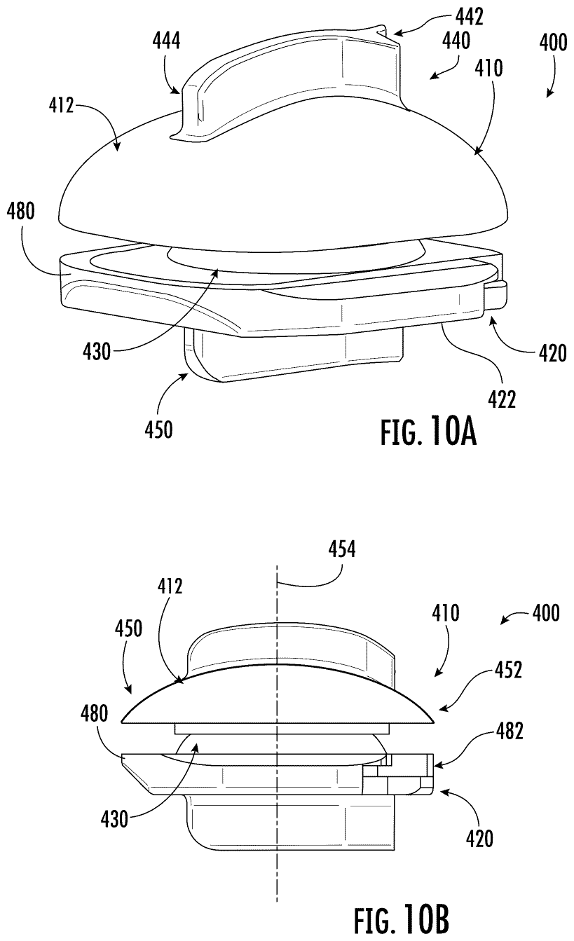

Referring now to FIGS. 10A and 10B, a spinal implant 400 comprises an upper or superior endplate 410, a lower or inferior endplate 420 and a core 430. Superior endplate 410 rides upon the core 430 and is operable to rotate relative to core 430, including torsional rotation about a vertical axis, flexion/extension rotation in the posterior/anterior directions and lateral bending rotation. Core 430 includes a substantially spherical dome 460 having a convex upper surface 465 that contacts an internal concave surface (not shown) of upper endplate 410 and an inlay 470 that couples dome 460 to lower endplate 420. The spherical dome 460 may be a separate element from the inlay 470 and coupled thereto, or the core and inlay may be manufactured as a single, integral element.

Endplates 410, 420 have a leading or posterior end 450, a trailing or anterior end 452 and an axis 454 extending through a midline of the endplates approximately halfway between posterior and anterior ends 450, 452. In this embodiment, core 430 is generally centered relative to posterior end 350 and anterior end 352. Alternatively, core 430 may be shifted posteriorly towards the posterior end 350, similar to the embodiment shown in FIGS. 1-3. In particular, spherical dome 460 has an apex that defines an axis or radius extending from the apex to a theoretical center of the spherical dome 460. The axis is substantially coincident with midline axis 454.

Similar to the embodiment shown in FIGS. 9A and 9B, upper endplate 410 has an upper bone contacting surface 412 that is substantially convex throughout the entire surface. Thus, surface extends, and is continuously convex, from a posterior end to an anterior end. In addition, surface 412 extends, and is continuously convex, from a first lateral end to second lateral end. Convex surface 412 has a first radius of curvature from the anterior end to the posterior end and a second radius of curvature from the first lateral end to the second lateral end. The second radius of curvature is greater than the first radius of curvature such that convex surface 412 extends downwardly towards lower endplate 420 more rapidly in the lateral direction than in the longitudinal direction. In addition, the convex surface 412 extends for a longer distance in the longitudinal direction than in the lateral direction.

In an exemplary embodiment, the ratio of the first radius of curvature to the second radius of curvature is about 1:2 to about 4:5, or about 2:3 to about 4:5. In certain embodiments the first radius of curvature may be between about spherical radius (SR) 5 mm to about SR 8 mm and the second radius of curvature may be between about SR 8 mm to about SR 10 mm for cervical applications. The first radius of curvature may be between about SR 14 mm to about SR 16 mm and the second radius of curvature may be between about SR 16 mm to about SR 20 mm for lumbar applications.

Similar to the embodiment shown in FIGS. 5A and 5B above, implant 400 may further include a recessed or sunken concave surface (not shown) of upper endplate 410 such that a greater portion of dome 460 resides within endplate 410 than resides between the endplates. As discussed below, this configuration allows upper endplate 410 to be disposed closer to lower endplate 420, which reduces the overall height of implant 400.

Implant 400 further includes fixation elements extending from each of the bone contacting surfaces 412, 422. These fixation elements may comprise one or more keels and/or one or more spikes and may have any of the configurations described herein. In the representative embodiment, implant 400 comprises a first keel 440 extending from upper bone contacting surface 412 of upper endplate 410 and a second keel 450 extending from lower bone contacting surface 422 of lower endplate 420. Keels 420, 440 are centrally located on endplates 410, 420 such that they generally extend along a longitudinal axis of the endplates.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, in a first aspect, a first embodiment is an implant for insertion into an intervertebral disc space between two adjacent vertebrae. The implant comprises a first component having an outer surface for engaging one of the adjacent vertebrae and a concave surface opposite the outer surface, a second component having an outer surface for engaging the other of the adjacent vertebrae and an inner surface opposite the outer surface and an articulating core member residing between the first and second components and comprising a semi-spherical protrusion with a convex surface in contact with the concave surface of the first component. The core member is eccentric to the first and/or the second component.

A second embodiment is the first embodiment, wherein the second component has a posterior end and an anterior end and a midline axis extending through the second component equally distant between the anterior and posterior ends and wherein the core member comprises an apex posterior to the midline axis of the second component.

A third embodiment is any combination of the above embodiments, wherein the apex is spaced about 1 mm to about 10 mm from the midline axis of the second component.

A 4^{th} embodiment is any combination of the above embodiments, wherein the apex is spaced about 2 mm to about 8 mm from the midline axis of the second component.

A 5^{th} embodiment is any combination of the above embodiments, wherein the convex surface defines a posterior surface area posterior to the midline axis of the second component and an anterior surface area anterior to the midline axis of the second component, wherein the posterior surface area is greater than the anterior surface area.

A 6^{th} embodiment is any combination of the above embodiments, wherein the posterior surface area is about 52% to about 80% of the anterior surface area.

A 7^{th} embodiment is any combination of the above embodiments, wherein the posterior surface area is about 55% to about 75% of the anterior surface area.

An 8^{th} embodiment is any combination of the above embodiments, wherein the first component has a posterior end and an anterior end and a midline axis extending through the first component equally distant between the anterior and posterior ends and wherein the apex of the convex surface is posterior to the midline axis of the first component.

A 9^{th} embodiment is any combination of the above embodiments, wherein the convex surface of the semi-spherical protrusion defines a radius from the apex to a center of the protrusion, wherein the radius is posterior of the midline axis of the second component.

A 10^{th} embodiment is any combination of the above embodiments, wherein the second component has a posterior end and the convex surface of the semi-spherical protrusion defines an arc extending from a posterior end to an anterior end, wherein the posterior end of the arc is about 1 mm to about 5 mm from the posterior end of the second component.

An 11^{th} embodiment is any combination of the above embodiments, wherein the second component has an anterior end and the convex surface of the semi-spherical protrusion defines an arc extending from a posterior end to an anterior end, wherein the anterior end of the arc is about 2 mm to about 8 mm from the anterior end of the second component.

A 12^{th} embodiment is any combination of the above embodiments, wherein the implant includes an upper endplate, and the outer surface of the upper endplate extends from an anterior end to a posterior end opposite the anterior end, wherein the outer surface is continuously convex from the anterior end to the posterior end.

A 13^{th} embodiment is any combination of the above embodiments, wherein the outer surface of the upper endplate extends from a first lateral end to a second lateral end opposite the first lateral end, wherein the outer surface is continuously convex from the first lateral end to the second lateral end.

A 14^{th} embodiment is any combination of the above embodiments, wherein the outer surface has a first radius of curvature from the anterior end to the posterior end and a second radius of curvature from the first lateral end to the second lateral end, wherein the first radius of curvature is less than the second radius of curvature.

A 15^{th} embodiment is any combination of the above embodiments, wherein the first component is an upper endplate and comprises a fixation element extending from the outer surface and wherein the second component is a lower endplate and comprises a fixation element extending from the outer surface.

A 16^{th} embodiment is any combination of the above embodiments, wherein the fixation element on the upper and lower endplates comprises one or more spikes.

A 17^{th} embodiment is any combination of the above embodiments, wherein the fixation element on the upper and lower endplates comprise one or more keels.

An 18^{th} embodiment is any combination of the above embodiments, wherein the articulating core member comprises an inlay, wherein the inlay is coupled to the second component and comprises a recess for receiving the semi-spherical protrusion.

A 19^{th} embodiment is any combination of the above embodiments, wherein the convex surface has a first portion with a first surface area in contact with the concave surface of the first component and a second portion with a second surface area between the first and second components, wherein a ratio of the first surface area to the second surface area is greater than about 1.4 to 1.

A 20^{th} embodiment is any combination of the above embodiments, wherein a surface area of the first portion is about 52% to about 80% of a surface area of the convex surface.

In another aspect, a first embodiment is an implant for insertion into an intervertebral disc space between two adjacent vertebrae. The implant comprises a first component having an outer surface for engaging one of the adjacent vertebrae and a concave surface opposite the outer surface, a second component having an outer surface for engaging the other of the adjacent vertebrae and an inner surface opposite the outer surface and an articulating core member residing between the first and second components and comprising a semi-spherical protrusion with a convex surface having a first portion with a first surface area in contact with the concave surface of the first component and a second portion with a second surface area between the first and second components. The ratio of the first surface area to the second surface area is greater than about 1.4 to 1.

A second embodiment is the first embodiment, wherein the ratio is greater than about 2 to 1.

A third embodiment is any combination of the above embodiments, wherein a surface area of the first portion is about 52% to about 80% of a surface area of the convex surface.

A 4^{th} embodiment is any combination of the above embodiments, wherein a surface area of the first portion is about 55% to about 75% of a surface area of the convex surface.

A 5^{th} embodiment is any combination of the above embodiments, wherein the first component is an upper endplate, and the second component is a lower endplate and the upper endplate comprises an inner surface facing the lower endplate.

A 6^{th} embodiment is any combination of the above embodiments, wherein the inner surface of the upper endplate comprises first and second portions, wherein the first portion extends at an angle relative to the second portion.

A 7^{th} embodiment is any combination of the above embodiments, wherein the first portion is a posterior portion of the inner surface and tapers away from the lower endplate in the posterior direction.

An 8^{th} embodiment is any combination of the above embodiments, wherein said angle is between about 3 degrees to about 12 degrees.

A 9^{th} embodiment is any combination of the above embodiments, wherein the upper endplate is configured to articulate relative to the lower endplate from a first position, wherein the second portion of the inner surface of the upper endplate is oriented at an angle less than about 20 degrees relative to the inner surface of the lower endplate, to a second position, wherein the second portion of the inner surface of the upper endplate is oriented at an angle greater than about 3 degrees relative to the inner surface of the lower endplate.

A 10^{th} embodiment is any combination of the above embodiments, wherein a maximum distance between the outer surfaces of the first and second endplates in the first position is about 12 mm or less.

An 11^{th} embodiment is any combination of the above embodiments, wherein said maximum distance is about 11 mm or less.

A 12^{th} embodiment is any combination of the above embodiments, wherein the posterior portion of the lower surface of the upper endplate contacts the inner surface of the upper endplate in the second position, wherein a minimum distance between the outer surfaces of the first and second endplates in the second position is less than about 8 mm.

A 13^{th} embodiment is any combination of the above embodiments, wherein said minimum distance is about 6 mm.

A 14^{th} embodiment is any combination of the above embodiments, wherein the first component comprises a fixation element extending from the outer surface and the second component comprises a fixation element extending from the outer surface.

A 15^{th} embodiment is any combination of the above embodiments, wherein the fixation element on the upper and lower endplates comprises one or more spikes.

A 16^{th} embodiment is any combination of the above embodiments, wherein the fixation element on the upper and lower endplates comprises one or more keels.

A 17^{th} embodiment is any combination of the above embodiments, wherein the articulating core member comprises an inlay, wherein the inlay is coupled to the second component and comprises a recess for receiving the semi-spherical protrusion.

In another aspect, a first embodiment is an implant for insertion into an intervertebral space between two adjacent vertebrae. The implant comprises a first component having an outer surface for engaging one of the adjacent vertebrae and an inner surface opposite the outer surface, the first component including a fixation element extending from the outer surface, a second component having an outer surface for engaging the other of the adjacent vertebrae and an inner surface opposite the outer surface, the second component including a fixation element extending from the outer surface, wherein the implant has an upper endplate and the outer surface of the upper endplate extends from an anterior end to a posterior end opposite the anterior end. The outer surface is substantially continuously convex from the anterior end to the posterior end.

A second embodiment is the first embodiment, wherein the outer surface of the upper endplate extends from a first lateral end to a second lateral end opposite the first lateral end, wherein the outer surface is substantially continuously convex from the first lateral end to the second lateral end.

A third embodiment is any combination of the above embodiments, wherein the outer surface has a first radius of curvature from the anterior end to the posterior end and a second radius of curvature from the first lateral end to the second lateral end, wherein the first radius of curvature is less than the second radius of curvature.

A 4^{th} embodiment is any combination of the above embodiments, wherein the first radius of curvature is substantially equal from the anterior end to the posterior end.

A 5^{th} embodiment is any combination of the above embodiments, wherein the second radius of curvature is substantially equal from the first lateral end to the second lateral end.

A 6^{th} embodiment is any combination of the above embodiments, wherein a ratio of the first radius of curvature to the second radius of curvature is about 1:2 to about 4:5.

A 7^{th} embodiment is any combination of the above embodiments, wherein the ratio is about 2:3 to about 4:5.

An 8^{th} embodiment is any combination of the above embodiments, wherein the fixation element of the first component comprises a spike and the fixation element of the second component comprises a keel.

A 9^{th} embodiment is any combination of the above embodiments, wherein the fixation element of the first component comprises a spike and wherein the fixation element of the second component comprises a first keel and a second keel.

A 10^{th} embodiment is any combination of the above embodiments, wherein the first component is an upper endplate and the second component is a lower endplate.

An 11^{th} embodiment is any combination of the above embodiments, wherein the outer surface of the lower endplate is substantially flat.

1. An implant for insertion into an intervertebral disc space between two adjacent vertebrae, the implant comprising:
a first component having an outer surface for engaging one of the adjacent vertebrae and a concave surface opposite the outer surface;
a second component having an outer surface for engaging the other of the adjacent vertebrae and an inner surface opposite the outer surface; and
an articulating core member residing between the first and second components and comprising a semi-spherical protrusion with a convex surface in contact with the concave surface of the first component, wherein the core member is eccentric to the first and/or the second component.

2. The implant of claim 1, wherein the second component has a posterior end and an anterior end and a midline axis extending through the second component equally distant between the anterior and posterior ends and wherein the core member comprises an apex posterior to the midline axis of the second component.

3. The implant of claim 2, wherein the apex is spaced about 1 mm to about 10 mm from the midline axis of the second component.

4. The implant of claim 2, wherein the convex surface defines a posterior surface area posterior to the midline axis of the second component and an anterior surface area anterior to the midline axis of the second component, wherein the posterior surface area is greater than the anterior surface area.

5. The implant of claim 5, wherein the posterior surface area is about 52% to about 80% of the anterior surface area.

6. The implant of claim 1, wherein the first component has a posterior end and an anterior end and a midline axis extending through the first component equally distant between the anterior and posterior ends and wherein the apex of the convex surface is posterior to the midline axis of the first component.

7. The implant of claim 6, wherein the convex surface of the semi-spherical protrusion defines a radius from the apex to a center of the protrusion, wherein the radius is posterior of the midline axis of the second component.

8. The implant of claim 1, wherein the second component has a posterior end and the convex surface of the semi-spherical protrusion defines an arc extending from a posterior end to an anterior end, wherein the posterior end of the arc is about 1 mm to about 5 mm from the posterior end of the second component.

9. The implant of claim 1, wherein second component has an anterior end and the convex surface of the semi-spherical protrusion defines an arc extending from a posterior end to an anterior end, wherein the anterior end of the arc is about 2 mm to about 8 mm from the anterior end of the second component.

10. An implant for insertion into an intervertebral disc space between two adjacent vertebrae, the implant comprising:
a first component having an outer surface for engaging one of the adjacent vertebrae and a concave surface opposite the outer surface;
a second component having an outer surface for engaging the other of the adjacent vertebrae and an inner surface opposite the outer surface,
an articulating core member residing between the first and second components and comprising a semi-spherical protrusion with a convex surface having a first portion with a first surface area in contact with the concave surface of the first component and a second portion with a second surface area between the first and second components, wherein a ratio of the first surface area to the second surface area is greater than about 1.4 to 1.

11. The implant of claim 10, wherein the ratio is greater than about 2 to 1.

12. The implant of claim 10, wherein the surface area of the first portion is about 52% to about 80% of a surface area of the convex surface.

13. The implant of claim 11, wherein the first component is an upper endplate, and the second component is a lower endplate, and the upper endplate comprises an inner surface facing the lower endplate, wherein the inner surface of the upper endplate comprises first and second portions, wherein the first portion extends at an angle relative to the second portion.

14. The implant of claim 13, wherein the first portion is a posterior portion of the inner surface and tapers away from the lower endplate in the posterior direction, and wherein said angle is between about 3 degrees to about 12 degrees.

15. An implant for insertion into an intervertebral space between two adjacent vertebrae, the implant comprising:
a first component having an outer surface for engaging one of the adjacent vertebrae and an inner surface opposite the outer surface, the first component including a fixation element extending from the outer surface;
a second component having an outer surface for engaging the other of the adjacent vertebrae and an inner surface opposite the outer surface, the second component including a fixation element extending from the outer surface; and
wherein the outer surface of the upper endplate extends from an anterior end to a posterior end opposite the anterior end, wherein the outer surface is substantially continuously convex from the anterior end to the posterior end.

16. The implant of claim 15, wherein the outer surface of the upper endplate extends from a first lateral end to a second lateral end opposite the first lateral end, wherein the outer surface is substantially continuously convex from the first lateral end to the second lateral end.

17. The implant of claim 16, wherein the outer surface has a first radius of curvature from the anterior end to the posterior end and a second radius of curvature from the first lateral end to the second lateral end, wherein the first radius of curvature is less than the second radius of curvature.

18. The implant of claim 17, wherein the first radius of curvature is substantially equal from the anterior end to the posterior end.

19. The implant of claim 17, wherein the second radius of curvature is substantially equal from the first lateral end to the second lateral end.

20. The implant of claim 17, wherein a ratio of the first radius of curvature to the second radius of curvature is about 1:2 to about 4:5.
